(19) United States
(12) Patent Application Publication
(10) Pub. No.: US 2011/0081715 A1
(43) Pub. Date: Apr. 7, 2011

(54) Single layer plastic test sample culture bottle

(75) Inventors: Ronnie J. Robinson, St. Charles, MO (US); Christopher S. Ronsick, Durham, NC (US); Mark S. Wilson, Hillsborough, NC (US)

(73) Assignee: bioMerieux Inc.

(21) Appl. No.: 12/893,161

(22) Filed: Sep. 29, 2010

Related U.S. Application Data
(60) Provisional application No. 61/278,159, filed on Oct. 2, 2009.

(51) Int. Cl. C12M 1/24; B05D 7/22; C23C 4/06; H05H 1/24

(52) U.S. Cl. 435/304.1; 427/230; 427/237; 427/452; 427/579

(57) ABSTRACT

A bottle for culturing a test sample, e.g., blood, includes a plastic vessel made from a single layer of plastic material. The bottle features a glass barrier coating applied to the bottle, such as a silica or glass coating. An alternative embodiment features a single layer plastic bottle and a gas barrier adhesive label covering the cylindrical side wall of the bottle. Kits comprising two or more of such bottles and methods of manufacturing the bottles are also disclosed.

### PRIORITY

This application claims priority benefits pursuant to 35 U.S.C. §119(e) to U.S. Provisional Application Ser. No. 61/278,159 filed Oct. 2, 2009, the content of which is incorporated by reference herein.

### BACKGROUND OF THE INVENTION

1. Field of the Invention

This invention relates to bottles for culturing test samples such as clinical test samples, e.g., blood, urine, or other biological specimens, and non-clinical test samples such as food. The culturing of the test sample can be for a variety of purposes, such as to detect or identify a microorganism present in the test sample or for quality control of the test sample.

2. Description of Related Art

Bottles for collection or culturing of blood and other biological samples are known in the art and described in the patent literature, see, e.g., U.S. Pat. Nos. 4,945,060; 5,094,955; 5,860,329; 4,827,944; 5,000,804; 7,211,430 and U.S. patent application publication 2005/0037165. Analytical instruments for analyzing the bottles for presence of organisms include U.S. Pat. Nos. 4,945,060; 5,094,955; 6,709,857 and 5,770,394, and WO 94/26874.

Blood culture bottles contain a specific headspace gas composition to ensure recovery of organisms. The blood culture container must be made of a suitable gas-impermeable material to ensure that the integrity of the gas composition in the headspace of the bottle is maintained throughout the shelf life of the bottle. The bottle should ideally remain transparent through its life for observation of the contents of the bottle, measuring fill level when using the bottle, for the user to visually observe contents after growth, and to enable reading of a sensor in the bottle that detects microbial growth.

Two types of blood culture bottles are currently used that limit gas diffusion into the bottle. One type is a glass vial with an elastomeric seal. The glass vial itself provides the gas barrier. However, glass has inherent safety risks. If a glass vial is dropped it is likely to break, exposing the user to glass shards and biologically hazardous materials. Furthermore, the nature of glass manufacturing can leave undetectable micro cracks in the glass, which under the pressure of microbial growth in the vial can lead to bottle rupturing, and exposing people to biohazardous materials. Accordingly, glass vials have drawbacks for use as blood culture bottles.

A second type of blood culture bottle is a multi-layer plastic vial. See, e.g., U.S. Pat. No. 6,123,211 and U.S. patent application publication 2005/0037165. The multi-layer plastic vial is fabricated from two plastic materials that each serve different functions. For example, the interior and exterior layers of the vials can be produced from polycarbonate, which offers the strength and rigidity required for product use. Likewise, polycarbonate can withstand higher temperatures required for autoclave of the product during manufacture and remains transparent. However, the polycarbonate does not provide a gas barrier. The middle material layer can be fabricated from nylon, which provides the gas barrier. The nylon, by itself, does not have the necessary rigidity and strength to withstand the autoclave temperatures required during the manufacture of blood culture bottles, since it would not remain transparent if exposed to moisture or autoclaved. The multilayer plastic vial offers advantages over the glass for safety. Another advantage is the reduced weight of the product. However, there are several drawbacks to multi-layer plastic vials, namely relatively complex manufacturing methods are required to manufacture the vials, and the vials are consequently relatively expensive. Furthermore, multi-layer plastic vials have environmental drawbacks, in that they cannot be recycled due to the presence of multiple materials. For example, set-up vials and scrapped vials when a faulty batch of bottles is manufactured cannot be ground up and reused for new bottles.

While the foregoing discussion has concentrated on issues relating to blood culture bottles, the invention is not limited to blood culture bottles. The methods and bottles of this disclosure can be used for culturing other types of test samples, including clinical and non-clinical test samples.

### SUMMARY

In one aspect, an improved bottle design for culturing a test sample is described herein which has the advantages of the multi-layer plastic vial (light weight, resistance to breakage) but with reduced product manufacturing complexity and cost. The bottle features a single plastic layer bottle or vial.

The gas barrier for the bottle is provided by one of several possible features. In one embodiment, a single layer plastic bottle is provided with a gas barrier coating, e.g., silica, or glass. The coating provides the gas barrier. The coating can be provided on either the exterior or interior surface of the bottle. The bottle can be made from suitable plastics such as nylon or polycarbonate which is autoclavable.

In still another aspect, a method of manufacturing a test sample culture device is contemplated, comprising the steps of: providing a single layer plastic bottle having an interior surface and an exterior surface; coating at least one of the interior and exterior surface of the bottle with a gas barrier coating; adding a growth media to the bottle; adding a specific headspace gas composition to the bottle; and placing a closure on the bottle. The gas barrier coating may take the form of silica or glass. The coating can be applied by a method selected from the group of methods consisting of: thermal spraying, plasma spraying, chemical vapor deposition and plasma-induced chemical vapor deposition.

In still another aspect, a device for culturing a test sample is described below in the form of a single layer plastic bottle containing a culture medium for promoting and/or enhancing growth of a microorganism present in a sample stored therein, the bottle having a cylindrical side wall, a bottom portion, and a neck portion; a gas barrier adhesive label applied to the cylindrical side wall of the single layer plastic bottle; and a closure for the bottle fitted to the neck portion. The gas barrier adhesive label may include a light blocking agent, such as a backing to the label. The adhesive label is made from a gas barrier material, such as EVOH, aluminum foil, aluminum foil/plastic laminate, or other suitable material. The label substantially completely covers the cylindrical side wall of the bottle but leaves the bottom, shoulder, and/or neck of the bottle exposed. Test sample culture kits are also contemplated including two or more devices for culturing a test sample, at least one of the devices is in the form of the single layer bottle with the gas-barrier adhesive label.

In still another aspect, a method of manufacturing a test sample culture device, is disclosed including the steps of: providing a single layer plastic bottle having a cylindrical side wall; adding a growth media to the bottle; adding a specific headspace gas composition to the bottle; placing a closure on the bottle having an exterior surface; and covering the cylindrical side wall with gas barrier adhesive label.

In the embodiments in which part of the single layer plastic bottle is exposed (such as the neck, bottom and shoulder portions of the bottle), some additional oxygen gas permeation occurs but the bottle still has a sufficient shelf life such that it can be used for microbiological testing purposes.

In one embodiment, a pair of blood culture bottles are shrink-wrapped together to form a testing kit ready for use for culturing test samples, such as blood samples. One of the bottles in the kit is configured with growth media for testing for the presence of aerobic microorganisms. The other bottle in the kit is configured with growth media for testing for the presence of anaerobic organisms. The shrink-wrap can be designed as a convenience to the user, for example the shrink-wrap could be perforated between bottles in the kit. Additionally, the kits could be configured in a continuous length of shrink-wrap and dispensed from a container, such as a box. The pair of bottles forming a test kit is dispensed from a box with a perforation in the shrink-wrap separating one pair of bottles from the next pair of bottles. The pair of bottles forming the test kit could also be dispensed one at a time. The packaging may also be designed to facilitate a “first in/first out” practice within the laboratory ensuring that the freshest bottles are used first and minimizing the risk of using a expired bottle. For example, the packaging (box) could be arranged where “new” bottles (or kits) are loaded into one end of the box and bottles are retrieved at an opposite end of the box.

Advantages for this design include reduction of manufacturing complexities of the multilayered vial. The bottles can for example be blow molded, a relatively inexpensive manufacturing process. An embodiment in which the barrier is made from the material EVOH (either shrink wrapped or in the form of an adhesive label) has significantly higher gas barrier properties than nylon. Moreover, the bottles of this disclosure are recyclable in that they are made from a single layer of plastic. Manufacturing defects in any bottles or bottles otherwise needing to be scrapped would be typically identified prior to application of the gas barrier shrink-wrap, adhesive label, or silica coating to the bottle. Such bottles can be ground up and turned into new bottles. This efficiency further reduces the costs of the bottles.

Current practice for blood culture bottles is to disinfect the stopper of the bottle before inoculation of the bottle with a patient's blood sample. Current blood culture bottle products have a removable plastic cap over the stopper. The plastic cap offers some mechanical protection of the stopper from damage and gross contamination, but the stopper is not sterile. The cap has to be removed prior to inoculation, and the stopper surface cleaned with a disinfectant, typically an alcohol wipe. In the shrink wrap embodiment in which the entire bottle is shrink-wrapped, the gas barrier material (shrink-wrap) encases the stopper, eliminating the need for the plastic cap and alcohol wipe, while also allowing for a sterile stopper.

### BRIEF DESCRIPTION OF THE DRAWINGS

Representative and non-limiting examples of embodiments of this invention are shown in the appended Figures, in which:

FIG. 1 is a perspective view of a blood culture bottle in accordance with this disclosure in which a single layer plastic blood culture bottle is completely enveloped in a gas barrier plastic shrink-wrap film.

FIG. 2 is an illustration of a continuous length of gas barrier shrink-wrap film enveloping multiple bottles of the type shown in FIG. 1.

FIG. 3 is an illustration of a kit of bottles, each of the type shown in FIG. 1, in which one of the bottles contains a growth medium for anaerobic microorganisms and the other bottle contains a growth medium for aerobic microorganisms.

FIG. 4 is a cross-section of the bottle of FIG. 3 along the lines 4-4.

FIG. 5 is an illustration of a dispensing container, e.g., box, that dispenses bottles of this disclosure, for example the bottle of FIG. 1, the kits of FIG. 4 or the continuous length of bottles as shown in FIG. 2.

FIG. 6 is a cross-section of a single layer plastic bottle with a gas barrier (e.g., silica or glass) coating on the interior surface of the bottle.

FIG. 7 is a cross-sectional view of a culture bottle featuring a gas barrier shrink-wrap covering the cylindrical side wall of the bottle, while leaving the bottom surface, neck and closure of the bottle exposed. The user does not have to remove the shrink wrap in order to use the bottle in this embodiment.

FIG. 8 is a cross-sectional view of a culture bottle having a gas barrier adhesive label applied to the cylindrical side wall of the bottle.

FIG. 9 is an elevation of the bottle of FIG. 8.

### DETAILED DESCRIPTION OF PREFERRED EMBODIMENTS

The following description will refer to a preferred embodiment of a culture bottle adapted for culturing a blood sample. However, the features and benefits of the disclosed embodiment are applicable to bottles for culturing clinical and non-clinical test samples generally, therefore the following description is offered by way of example and not limitation. All questions concerning scope of the invention should be answered by reference to the appended claims.

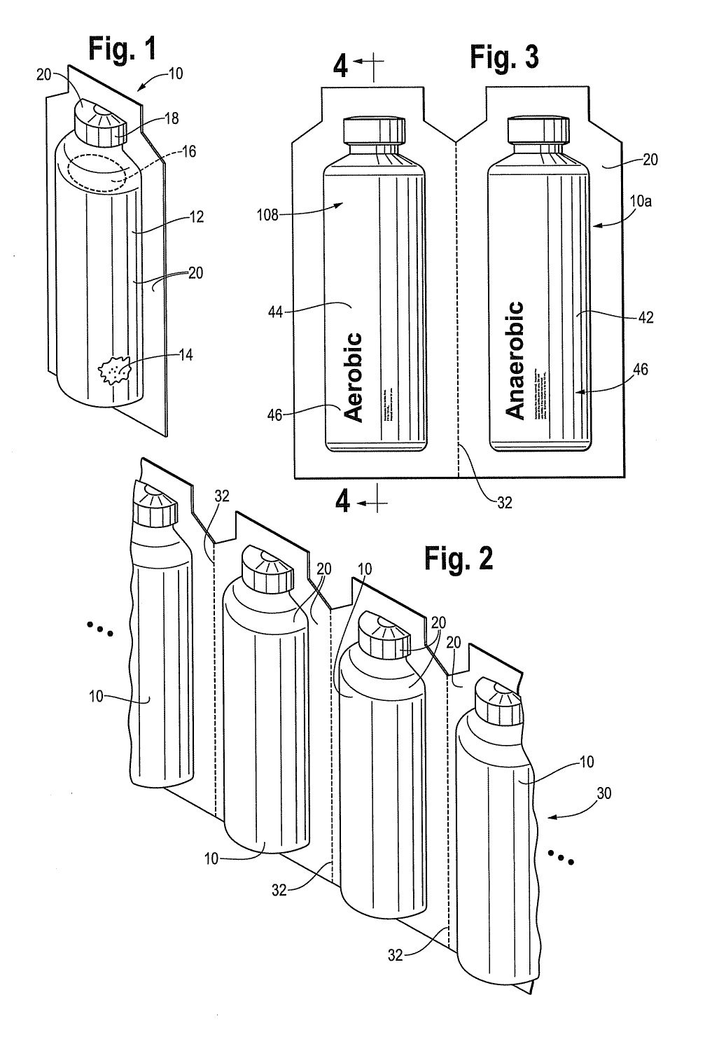

FIG. 1 is a perspective view of a blood culture device 10 in accordance with one aspect of this disclosure. The device includes a plastic vessel or bottle 12 which is made from a single layer of plastic material. The plastic material used to form vessel or bottle 12 preferably meets two requirements: unaffected by high temperatures occurring during autoclaving, and light transmittance (bottle is made from a transparent material) in order for reading of a colorimetric sensor in the bottle. Preferred embodiments use blow molding for forming the bottle. Other types of techniques for manufacture of the bottle are also possible. The bottle should have the necessary strength characteristics and ability to be autoclaved, hence transparent polycarbonate is a preferred material for the bottle. Other useful plastic may include polypropylene (PP), polyethylene terephthalate (PET), polyethylene napthalate (PEN) or other well known materials in the plastics art. Amorphous plastics such as amorphous nylon exhibit high transparency and may be suitable if they are able to withstand autoclaving. The vessel 12 contains a growth media 14 for culturing a microorganism within the bottle and has a headspace 16 having a desired or specific gas composition. The gasses in the headspace 16 are introduced into the bottle during manufacture. The bottle 10 further includes a closure 18 for the plastic vessel 12, such as a stopper. The vessel 12 is autoclaved after introduction of the growth media 14 and the headspace gas composition and fitting of the closure 18, thereby sterilizing the vessel 12 including the exterior surface of the closure 18.

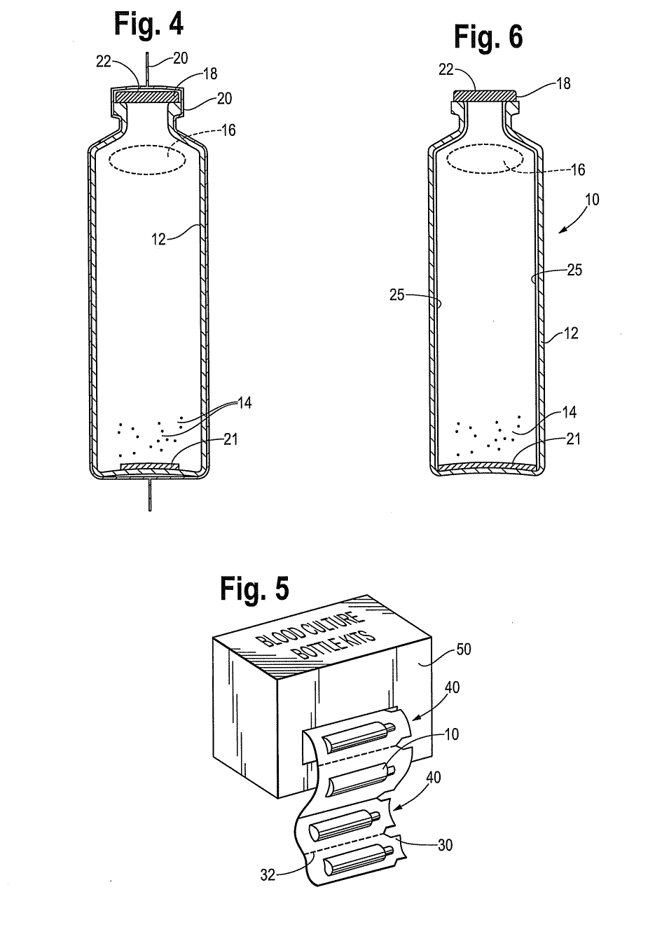

In the embodiment of FIGS. 1-5, the bottle 12 further includes a removable, gas barrier plastic shrink-wrap film 20 completely enveloping the plastic vessel 12, thereby maintaining the integrity of the gas composition in the headspace 16. The shrink-wrap 20 further completely envelops the closure 18. The shrink-wrap 20 is best shown in the cross-sectional view of FIG. 4. The gas barrier plastic shrink-wrap 20 may take the form of an Ethylene-Vinyl Alcohol Copolymer plastic shrink-wrap in one embodiment. Alternative plastic gas barrier materials may include, for example, polyester, nitrile barrier resins, polyvinyl chloride, polyamides, polyvinylidene chloride, polyvinylidene chloride coated polyethylene, polyvinylidene chloride coated polyester, and polyvinylidene chloride coated polyamide films. The shrink-wrap 20 is completely removed from the bottle at the time of use to expose the stopper or closure 18 for the vessel 12 to the user and allow the blood sample to be introduced into the interior of the vessel 12. Also, if the bottle includes a colorimetric or fluorescence sensor 21, the removal of the shrink-wrap from the bottle may advisable so as to not interfere with the reading of the sensor 21 by a detection instrument. The sensor 21 is shown schematically and may take different forms or shapes or be located at different positions within the bottle, the details of which are not important.

The closure 18 has an exterior surface 22 (FIG. 4) which is sterilized prior to being enveloped in the shrink-wrap 20. In this manner, when the shrink-wrap 20 is removed the bottle can be immediately used without requiring a separate step of wiping the surface 22 of the stopper with alcohol.

FIG. 2 is an illustration of a continuous length 30 of gas barrier shrink-wrap film 20 enveloping multiple bottles 10, each of the type shown in FIG. 1. The length 30 of film 20 includes perforations 32 which, when torn, separate each bottle from an adjacent bottle.

FIG. 3 is an illustration of a blood culture kit 40 comprising two culture devices 10A and 10B of the type shown in FIG. 1. The devices are shown in cross-section in FIG. 4 and are of identical construction. The kit 40 includes a first plastic vessel 42 made from a single layer of plastic material for receiving a first blood sample and containing a growth media for an anaerobic organism and having a headspace; a closure 18 (FIG. 4) for the first plastic vessel; a second plastic vessel 44 made from a single layer of plastic material for receiving a second blood sample and containing a growth media for an aerobic organism and having a headspace; a closure 18 for the second plastic vessel; and a gas barrier plastic shrink-wrap 20 completely enveloping the first and second single layer plastic vessels 42 and 44 as a unit. In one embodiment, the gas barrier plastic shrink-wrap includes a perforation 32 for separating the first and second devices 10A and 10B from each other. In preferred embodiments, the first and second plastic vessels 42 and 44 are in the form of blow-molded plastic bottles, such as, for example, blow-molded transparent polycarbonate. The gas barrier plastic shrink-wrap 20 may take the form of an Ethylene-Vinyl Alcohol Copolymer plastic shrink-wrap. The gas barrier plastic shrink-wrap completely envelops the closure 18 as shown in FIG. 4. The closure 18 of each of the first and second bottles is sterilized prior to being enveloped in the gas barrier plastic shrink-wrap.

Printing 46 is applied to the shrink-wrap 20 to identify the bottle type. Additional label information could be added to the bottle shrink-wrap via the printing 46, thereby reducing the label size for the bottles per se and providing additional space on the bottle for customer-applied labels.

The user completely removes the shrink-wrap from the bottles, and introduces one sample from the subject into the bottle 42 and another sample from the subject into the bottle 44. The bottles 42 and 44 could be separated from each other by perforations in the shrink-wrap as indicated at 32.

FIG. 5 is an illustration of a dispensing container, e.g., box 50, that dispenses culture devices 10 of this disclosure, for example the device 10 of FIG. 1, the kits 40 of FIG. 4 or the continuous length 30 of devices 10 as shown in FIG. 2.

With reference to FIGS. 1-4, in another aspect, a method of manufacturing a blood culture device, comprising the steps of:

providing a single plastic layer bottle 12;

adding a growth media 14 to the bottle;

adding a specific headspace gas composition 16 to the bottle;

placing a closure 18 on the bottle having an exterior surface 22 (FIG. 4);

sterilizing the exterior surface 22 of the closure 18 (e.g., via autoclaving); and

completely enveloping the bottle 12 and closure 18 in a gas barrier plastic shrink-wrap 20.

In another aspect, a method of manufacturing a blood culture kit is contemplated, comprising the steps of:

completely enveloping an anaerobic blood culture bottle 42 and an aerobic blood culture bottle 44 in a gas barrier shrink-wrap 20 to form a unit of the bottles enveloped in the shrink-wrap (FIGS. 3 and 4); wherein the anaerobic blood culture bottle and the aerobic blood culture bottle are made from a single plastic layer, such as for example blow molded polycarbonate.

The method optionally further comprises the step of sterilizing the exterior surface 22 of the closure 18 for the first and second bottles, e.g., using autoclaving.

The method may further comprise the step of perforating the gas barrier plastic shrink-wrap 22 between the aerobic and anaerobic bottles as indicated at 32 in FIG. 3.

The method may further comprise the step of forming a continuous length of the kits as shown in FIGS. 2 and 5 in a length 30 of gas barrier plastic shrink-wrap 20, and, forming a perforation in the length of gas barrier plastic shrink-wrap to facilitate separation of one kit in the continuous length from another, as indicated in FIG. 5 with the perforations 32. The method may also include the step of placing the continuous length 32 of the kits 40 into a dispensing container, e.g., dispensing box or pouch 50. In one embodiment, the box is configured such that it facilitates first in/first out laboratory practices, such as providing an opening at one end of the box for introduction of new bottles or kits, and a second opening at the opposite end for removal of bottles or kits by the users, with the bottles or kits advancing progressively through the dispensing container in a first in/first out fashion. The dispensing device could take the form of a display-type container such as used in the vending, art.

The contents (growth medium 14) in the bottles 12 should be protected from light. The shrink-wrap could include a light barrier, e.g., aluminum foil backing or blocking agent in the plastic material to protect the contents from photo degradation.

Occasionally, a blood culture bottle will leak at the bottle closure 18. The integrity of this primary seal is enhanced by the shrink-wrap 20.

One of the uses of the bottles of this disclosure is in performing a method for culturing a test sample to detect microbial growth in test sample (e.g., a blood sample) suspected of containing a microorganism therein. The method includes a step of (a) providing a specimen container (device 10) including a culture medium 14 for promoting and/or enhancing growth of the microorganism, wherein the specimen container comprises: (i) a plastic vessel 12 made from a single layer of plastic material; (ii) a closure 18 for the plastic vessel; and (iii) a removable, gas barrier plastic shrink-wrap 20 completely enveloping the plastic vessel 12; (b) removing the gas barrier plastic shrink-wrap; (c) inoculating the specimen container 10 with the test sample; (d) incubating the specimen container with a test sample to be tested for the presence of a microorganism (e.g., by placing the bottle in an incubation instrument); and (e) monitoring the specimen container for microorganism growth, either manually or automatically using a sensor.

Partially Shrink-Wrapped Bottle

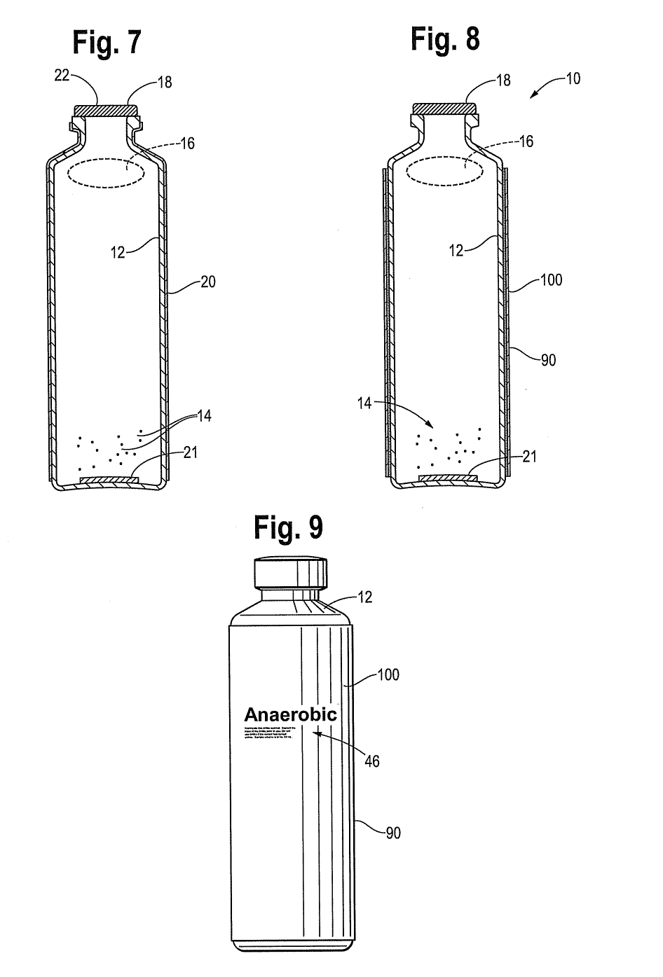

A variation of the design of FIGS. 1-5 provides for a partially shrink-wrapped bottle. This embodiment is shown in FIG. 7. The cylindrical side walls and neck of the bottle 12 are enveloped in a gas barrier shrink-wrap 20, but the bottom surface of the bottle (below the colorimetric sensor 21) and the area around the periphery of the stopper 18 are not covered in shrink-wrap. The user does not have to remove the shrink-wrap 20 at the time of use. Rather, they clean the exterior surface 22 of the stopper 18, inoculate the bottle 12 with the specimen, and place the bottle into an incubation and detection instrument. The absence of the shrink-wrap in the area below the sensor 21 insures that the shrink-wrap does not interfere with the measurements of the colorimetric sensor 21 in the instrument. The absence of the gas barrier shrink-wrap in the region below the sensor 21 and the small portion at the very upper end of the bottle 12 may permit some ingress of oxygen gas into the interior of the bottle at these locations, but the amount of oxygen gas intrusion into the bottle is so small that the bottle will normally have sufficient shelf life in which the specifications for the composition of the head-space gasses 16 are within design limits, particularly in the case of culture bottles designed for detection of aerobic microorganisms.

The gas barrier plastic shrink-wrap 20 in the embodiment of FIG. 7 may take the form of an Ethylene-Vinyl Alcohol Copolymer plastic shrink-wrap, optionally with a light barrier, e.g., aluminum foil backing or opaque/blocking agent incorporated in the shrink-wrap material.

Bottles of the design of FIG. 7 can be grouped in pairs to form a kit as described in conjunction with FIGS. 3 and 5.

The material for the bottle 12 is preferably optically clear, autoclavable plastic such as polycarbonate.

One of the uses of the bottles of FIG. 7 is in performing a method for culturing a test sample to detect microbial growth in test sample (e.g., a blood sample) suspected of containing a microorganism therein. The method includes a step of (a) providing a specimen container (device 10) including a culture medium 14 for promoting and/or enhancing growth of the microorganism, wherein the specimen container comprises: (i) a plastic vessel 12 made from a single layer of plastic material; (ii) a closure 18 for the plastic vessel; and (iii) a removable, gas barrier plastic shrink-wrap 20 partially enveloping the plastic vessel 12; (b) inoculating the specimen container 10 with the test sample; (c) incubating the specimen container with a test sample to be tested for the presence of a microorganism (e.g., by placing the bottle in an incubation instrument); and (d) monitoring the specimen container for microorganism growth, either manually or automatically using a sensor.

Single Layer Plastic Bottles without Shrink-Wrap

In still another aspect of this disclosure, single layer plastic bottles 12 are contemplated for use in culturing a test sample, in which there is no need for a shrink-wrap gas barrier layer 20 as shown in FIGS. 1-4 and 7. In accordance with this embodiment, the single layer plastic bottle or vessel 12 itself will have properties of gas impermeability, transparency, strength, and ability to be autoclaved without loss of transparency. In general, any known plastic material that provides these properties can be used in the practice of this embodiment. For example, Grivory® Nylon FE 7105 (available from EMS-Grivory (North America) Inc., Sumter S.C.) may be used for the vessel 12. The vessel 12 may be manufactured from this plastic by suitable methods such as blow molding. A growth media 14 is contained within the bottle for culturing a microorganism. The bottle 10 includes a closure 18 and a headspace 16 in the bottle having a desired gas composition. Such bottles can be used for the kits of this disclosure, packaged in pairs as disclosed above using any convenient shrink wrap which only serves a purpose of a joining pairs of bottles as a unit.

Adhesion promotors for adhering a liquid emulsion colorimetric sensor 21 to the interior of the bottle may be needed with bottles made in accordance with this embodiment.

Single Layer Plastic Bottles with Gas Barrier Coating

In yet another aspect of this disclosure, as shown in FIG. 6, a culture device 10 includes a vessel or bottle 12 made from a single layer of plastic material which is coated with a gas barrier material shown as coating 25. For example, a single layer polycarbonate bottle 12 can be coated with a silica or glass layer 25 to provide a gas barrier. Other coatings 25 that provide a gas barrier may also be used, and may include, for example, a metal coating layer, a ceramic coating layer, or a gas barrier plastic coating layer. In one embodiment, the interior wall of the bottle is coated as shown in FIG. 6. The exterior of the bottle could be coated either in addition to coating on the interior of the bottle, or as an alternative to coating on the interior.

The bottle can be coated with silica or glass by known means in the art. For example, the coating 25 can be applied by thermal spraying, plasma spraying or chemical vapor deposition. A silica coating can be applied by plasma-induced chemical vapor deposition. This method may employ high frequency energy in combination with hexamethyl disiloxane in an oxygen-rich environment to result in deposition of silica (SiO₂) on the inner surface of the bottle. In accordance with this embodiment, there is no need to shrink-wrap the bottle 10 of FIG. 6 to provide a gas barrier. However, in an alternative aspect, the bottle may further comprise a shrink-wrap barrier, as disclosed hereinabove and shown in FIG. 4 or FIG. 7, e.g., after autoclaving the bottle to preserve sterility on the exterior surface 22 of the closure 18. As with other embodiments disclosed herein, the coated single layer bottle 12 can be used in a method for culturing and/or for detecting growth of a microorganism in a test sample (e.g., a blood sample). Again, the monitoring step may be performed manually or automatically, e.g., via monitoring a colorimetric sensor located within the bottle for a color change indicative of microorganism growth as described in U.S. Pat. Nos. 4,945,060 and 5,094,955.

Single Layer Plastic Bottle with Gas Barrier Labels

A further embodiment of a single layer plastic bottle 10 with a gas barrier is shown in FIGS. 8 and 9. In this embodiment, the gas barrier is in the form of an adhesive label 100 which is applied to the cylindrical side wall 90 of the bottle 12. The adhesive label 100 is made from a gas barrier material such as Ethylene-Vinyl Alcohol Copolymer, optionally including a light barrier, e.g., aluminum foil backing or opaque/blocking agent incorporated in the label material 100. The label is sized so as to substantially completely cover the cylindrical side wall 90 of the bottle 12 as shown in FIGS. 8 and 9, leaving the bottom of the bottle and the neck/cap area uncovered. As with the case with the partially shrink-wrapped bottle of FIG. 7, some gas permeation into the interior of the bottle is to be expected due to the bottle 12 not being completely covered by a gas barrier material, but the rate of gas ingress is sufficiently slow that the bottle will have an acceptable shelf life during which the composition of the headspace gasses 16 are within design limits.

The label 100 includes printing 46 as shown in FIG. 9, e.g., identifying the type of microorganism the bottle is to be used to culture, lot number, expiration date, bar codes, or other matter.

Kits for culturing test samples may include one or more of the bottles as shown in FIGS. 8 and 9. For example, the kit may take the form of a blood culture kit having two bottles, one of which is an anaerobic blood culture bottle and the other of which is an aerobic blood culture bottle. The aerobic blood culture bottle includes the gas barrier adhesive label as shown in FIGS. 8 and 9. The anaerobic blood culture bottle could take the form of the shrink-wrapped bottle of FIG. 4, or a bottle with the gas barrier coating as shown in FIG. 4, or a bottle as shown in FIGS. 8 and 9.

A method of manufacturing a test sample culture device is contemplated for the design of FIGS. 8 and 9, comprising the steps of: providing a single layer plastic bottle 12 having a cylindrical side wall; adding a growth media 14 to the bottle; adding a specific headspace gas composition 16 to the bottle; placing a closure 18 on the bottle; and covering the cylindrical side wall 90 with gas barrier adhesive label 100.

Further Considerations

In general, and without in any way limiting the scope of the invention, the gas permeation rate of any monolayer plastic bottle with a gas barrier (partial or full gas barrier shrink-wrap, gas barrier coating, or gas barrier adhesive label as in this disclosure) may be non-zero. That is, some ingress of oxygen gas occurs despite the presence of the gas barrier shrink wrap, gas barrier coating, or gas barrier adhesive label. For some existing multi-layer plastic bottles (prior art), the gas permeation rate is approximately 0.0038 cc/bottle per day for oxygen gas. Ideally, the gas permeation rate for any of the embodiments of this disclosure approximates or exceeds this rate.

Initial testing of single layer plastic bottles with a silica coating on the interior of the bottle (FIG. 6) has shown a gas permeation rate is between 0.003 and 0.005 cc/bottle per day, which is considered encouraging in that it is close to the rate of existing bottles. The rate may be reduced by changing the recipe for the silica coating or changing the thickness of the silica coating.

The gas permeation rate for the single layer bottle made from EMS Grivory FE-7105 was tested and resulted in a rate that was, approximately twice the rate of existing (prior art) multi-layer plastic bottles. The additional oxygen may affect anaerobic products and result in a shorter shelf life for such bottles. The nylon formula for EMS Grivory 7105, or the wall thickness of the bottle, may be optimized to decrease the gas permeation rate.

The gas permeation rates for the gas barrier shrink-wrapped bottles and bottles having gas-barrier adhesive labels will depend on the material used for the shrink-wrap and the label, the thickness of such material, and the extent to which it covers the monolayer plastic bottle (either completely or nearly so as in FIG. 7). Persons skilled in the art will be able to optimize such parameters to meet design objectives for gas permeation rate, e.g., 0.003 to 0.005 cc/bottle per day, and if necessary adjust the shelf life or expiration dates to meet design requirements.

We claim:

1. A device for culturing a test sample, comprising:
a single layer plastic bottle containing a culture medium for promoting and/or enhancing growth of a microorganism present in a sample stored therein;
a gas barrier coating applied to said single layer plastic bottle; and
a closure for the bottle.

2. The device of claim 1, wherein the gas barrier coating comprise a silica coating.

3. The device of claim 1, wherein the oxygen gas barrier coating comprises a glass coating.

4. The device of claim 2, wherein said single layer plastic bottle comprises an interior wall and an exterior wall, and wherein said interior wall is coated with the silica coating.

5. The device of claim 2, wherein said single layer plastic bottle comprises an interior wall and an exterior wall defining the exterior of the bottle, and wherein said exterior wall is coated with the silica coating.

6. The device of claim 3, wherein said single layer plastic bottle comprises an interior wall and an exterior wall defining the exterior of the bottle, and wherein said interior wall is coated with the glass coating.

7. The device of claim 3, wherein said single layer plastic bottle comprises an interior wall and an exterior wall defining the exterior of the bottle, and wherein said exterior wall is coated with the glass coating.

8. The device of claim 1, wherein the single layer plastic bottle comprises a transparent polycarbonate bottle.

9. A method of manufacturing a test sample culture device, comprising the steps of:
providing a single layer plastic bottle having an interior surface and an exterior surface;
coating at least one of the interior and exterior surface of the bottle with a gas barrier coating;
adding a growth media to the bottle;
adding a specific headspace gas composition to the bottle; and
placing a closure on the bottle.

10. The method of claim 9, wherein the coating step comprises coating the interior surface of the bottle.

11. The method of claim 9, wherein the coating is applied by a method selected from the group of methods consisting of: thermal spraying, plasma spraying, chemical vapor deposition and plasma-induced chemical vapor deposition.

12. The method of claim 9, wherein the single layer plastic material comprises transparent polycarbonate.

13. The method of claim 9, further comprising the step of autoclaving the bottle and an exterior surface of the closure and, subsequently enveloping the bottle and the closure in a removable plastic shrink-wrap.

14. The method of claim 9, wherein the bottle is formed from the single layer plastic material using blow molding.

15. A test sample culture kit comprising two or more bottles as manufactured in accordance with the method of claim 9.

16. The kit of claim 15, wherein the kit comprises a blood culture kit and wherein the kit comprises two bottles, one of which is an anaerobic blood culture bottle and the other of which is an aerobic blood culture bottle.

17. A device for culturing a test sample, comprising:
a single layer plastic bottle containing a culture medium for promoting and/or enhancing growth of a microorganism present in a sample stored therein, the bottle having a cylindrical side wall, a bottom portion, and a neck portion;
a gas barrier adhesive label applied to the cylindrical side wall of the single layer plastic bottle; and
a closure for the bottle fitted to the neck portion.

18. The device of claim 17, wherein the gas barrier adhesive label includes a light blocking agent.

19. The device of claim 18, wherein the light blocking agent comprises a backing to the label.

20. The device of claim 17, wherein the single layer plastic bottle comprises blow-molded, clear polycarbonate bottle.

21. The device of claim 17, wherein the bottle further comprises a colorimetric sensor incorporated into the interior of the bottle adjacent to the bottom portion of the bottle.

22. A test sample culture kit comprising two or more devices for culturing a test sample, at least one of the devices comprising device as set forth in claim 17.

23. The kit of claim 22, wherein the kit comprises a blood culture kit and wherein the kit comprises two bottles, one of which is an anaerobic blood culture bottle and the other of which is an aerobic blood culture bottle, wherein the aerobic blood culture bottle comprises the device as set forth in claim 17.
